(19) United States
(12) Patent Application Publication
(10) Pub. No.: US 2024/0277583 A1
(43) Pub. Date: Aug. 22, 2024

(54) Micellar Mouthwash Composition

(71) Applicant: Colgate-Palmolive Company, New York, NY (US)

(72) Inventors: Changlong CHEN, New Brunswick, NJ (US); Chi-Yuan Cheng, Hillsborough, NJ (US); Hongwei Shen, Holmdel, NJ (US); Zhigang Hao, Bridgewater, NJ (US); Svenia Schmid, Basel (CH); Thomas Schollbach, Basel (CH); Cornelia Scheffel, Aesch (CH)

(21) Appl. No.: 18/512,294

(22) Filed: Nov. 17, 2023

Related U.S. Application Data
(60) Provisional application No. 63/426,111, filed on Nov. 17, 2022.

(51) Int. Cl. A61K 8/02; A61K 8/41; A61Q 11/00

(52) U.S. Cl. CPC A61K 8/0291; A61K 8/41; A61Q 11/00; A61K 2800/30; A61K 2800/92

(57) ABSTRACT

The present disclosure relates to a mouthwash composition containing an amine fluoride or amine base plus fluoride and an alkyl glucoside, as well as related methods for use of said compositions.

### FIELD

The present disclosure relates to micellar mouthwash compositions containing an amine fluoride and an alkyl glucoside, as well as related methods for use of said compositions.

### BACKGROUND

Fluoride is one of the agents commonly used to combat dental caries. Fluoride can greatly help dental health by strengthening the tooth enamel, making it more resistant to tooth decay. Fluoride also helps remineralize weakened tooth enamel and reverses early signs of tooth decay. Many oral care products such as toothpastes, mouthwashes and professional dental treatment products contain fluoride. Amine fluorides are well known fluoride ion sources used in oral care products. The amine base has a hydrophobic non-polar tail and a hydrophilic polar amine head. Therefore, amine fluorides act like surfactants. Due to their surface activity, amine fluorides form a thin hydrophobic film on the tooth enamel on oral use of the hygiene composition. Thus, on the one hand, the tooth enamel becomes more resistant to acid attacks on account of the CaF₂ covering layer formed. On the other hand, the long-chain hydrocarbon residues form a hydrophobic layer which prevents the formation of deposits and the attack of the acidic degradation products on the tooth enamel.

A micelle is an aggregate of surfactants dispersed in a liquid, forming a colloidal suspension. Micelles provide dual benefits in a product. Micelles deliver lipophilic actives like essential oil and also act to enhance cleaning efficiency via encapsulating the dirt.

There is a need for mouthwash formulations containing amine fluorides with improved micellar properties.

### BRIEF SUMMARY

In an aspect, the invention provides a mouthwash composition comprising an amine fluoride or amine base plus fluoride and an alkyl glucoside. In certain embodiments, the amine fluoride is olaflur (N′-octadecyltrimethylenediamine-N,N,N′-tris(2-ethanol)-dihydrofluoride).

In some embodiments, the amine fluoride is present in an amount of from 0.5% to 1.5%, e.g., from 0.5% to 1.2%, from 0.5% to 1%, from 0.7% to 1.5%, from 0.7% to 1.2%, from 0.7% to 1%, from 0.8% to 1.5%, from 0.8% to 1.2%, from 0.8% to 1%, from 0.85% to 0.95%, or about 0.9% by weight of the composition.

The alkyl glucoside may be C₈₋₂₅ alkyl glucoside, e.g., C₈₋₁₈ alkyl glucoside, or C₁₀₋₁₈ alkyl glucoside. In some embodiments, the alkyl glucoside is selected from coco glucoside, decyl glucoside, caprylyl/capryl glucoside, lauryl glucoside, coco glucoside, octyl glucoside, cetearyl glucoside, cetyl glucoside, hexadecyl glucoside, arachidyl glucoside, and a combination thereof. In some embodiments, the alkyl glucoside is selected from coco glucoside, decyl glucoside, caprylyl/capryl glucoside and a combination thereof. In certain embodiments, the alkyl glucoside is coco glucoside.

In some embodiments, the alkyl glucoside is present in an amount of from 0.1% to 2.5%, e.g., from 0.1% to 1%, from 0.1% to 0.6%, from 0.1% to 0.5%, from 0.1% to 0.4%, from 0.1% to 0.3%, from 0.2% to 0.4%, from 0.2% to 0.3%, from 0.25% to 0.3%, or about 0.27% by weight of the composition.

In another aspect, the invention provides a method comprising applying an effective amount of any of mouthwash compositions as disclosed herein to the oral cavity to a subject in need thereof, to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity.

In another aspect, the invention provides the use of an alkyl glucosides in a mouthwash composition comprising an amine fluoride, e.g., any of mouthwash compositions as disclosed herein for improving micellar properties of the mouthwash composition, e.g., decreasing critical micelle concentration (CMC) of the amine fluoride in the composition and/or decreasing micelle (colloid particle) size.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### BRIEF DESCRIPTION OF FIGURES

FIG. 1 depicts LC-HRMS data demonstrating an amine base-coco glucoside complex (indicated by an arrow).

FIG. 2 depicts LC-HRMS data demonstrating an amine base-coco glucoside complex (indicated by an arrow).

### DETAILED DESCRIPTION

The following description of various typical aspect(s) is merely exemplary in nature and is in no way intended to limit the disclosure, its application, or uses.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight. The amounts given are based on the active weight of the material.

In the present invention, it has been found that the use of an alkyl glucoside as a surfactant improves micellar properties of mouthwash formulations containing an amine fluoride (e.g., olaflur (N′-octadecyltrimethylenediamine-N,N,N′-tris(2-ethanol)-dihydrofluoride). The combination of an amine fluoride and coco-glucoside has been found to decrease critical micelle concentration (CMC) of the amine fluoride and decreasing micelle (colloid particle) size, compared to amine fluoride/PEG-40 hydrogenated castor oil. Low critical micelle concentration of amine fluoride/cocoyl glucoside mixture ensures higher population of micelles in the mouthwash. Smaller micelle size of the amine fluoride/cocoyl glucoside complex exhibits higher mobility, which is likely to enhance active delivery and antibacterial efficacy in the mouthwash. The 2D NOESY and dynamic light scattering data suggest strong interaction between amine fluoride and coco-glucoside. Without intending to be bound to any theory, the improved micelle properties may be due to strong synergistic interaction between cationic and nonionic surfactants having comparable molecular size. Thus, the amine fluoride/cocoyl glucoside combination is an improved alternative to amine fluoride/PEG-40 hydrogenated castor oil to form micelles in mouthwash formulations.

The present invention provides, in an aspect, a mouthwash composition (Composition 1.0) comprising an amine fluoride or amine base plus fluoride and an alkyl glucoside.

For example, the invention includes:
1.1. Composition 1.0, wherein the alkyl glucoside is C₈₋₂₅ alkyl glucoside, e.g., C₈₋₁₈ alkyl glucoside, or C₁₀₋₁₈ alkyl glucoside.
1.2. Any of the preceding compositions, wherein the alkyl glucoside is selected from coco glucoside, decyl glucoside, caprylyl/capryl glucoside, lauryl glucoside, octyl glucoside, cetearyl glucoside, cetyl glucoside, hexadecyl glucoside, arachidyl glucoside, and a combination thereof.
1.3. Any of the preceding compositions, wherein the alkyl glucoside is selected from coco glucoside, decyl glucoside, caprylyl/capryl glucoside and a combination thereof.
1.4. Any of the preceding compositions, wherein the alkyl glucoside is coco glucoside.
1.5. Any of the preceding compositions, wherein the alkyl glucoside is present in an amount of from 0.1% to 2.5%, e.g., from 0.1% to 1%, from 0.1% to 0.6%, from 0.1% to 0.5%, from 0.1% to 0.4%, from 0.1% to 0.3%, from 0.2% to 0.4%, from 0.2% to 0.3%, from 0.25% to 0.3%, or about 0.27% by weight of the composition.
1.6. Any of the preceding compositions, wherein the amine base is a primary amine, secondary amine, tertiary amine or a combination thereof.
1.7. Any of the preceding compositions, wherein the amine base comprises or consists of a primary amine base.
1.8. Any of the preceding compositions, wherein the amine base comprises or consists of a secondary amine base.
1.9. Any of the preceding compositions, wherein the amine base comprises or consists of a tertiary amine base.
1.10. Any of the preceding compositions, wherein the amine base is plant-derived.
1.11. Any of the preceding compositions, wherein the amine base is animal-derived.
1.12. Any of the preceding compositions, wherein the amine base is derived from bovine tallow.
1.13. Any of the preceding compositions, wherein the amine base is derived from rapeseed oil or from rice bran oil.
1.14. Any of the preceding compositions, wherein the amine base is a linear or branched fatty amine or polyamine, or mixtures thereof.
1.15. The preceding composition, wherein the amine base is a saturated or unsaturated C₁₂₋₂₀ alkyl amine base or a saturated or unsaturated C₁₂₋₂₀ alkyl polyamine base, or mixtures thereof.
1.16. Any of the preceding compositions, wherein the amine base is a myristyl, palmityl, linoleyl, oleyl, or stearyl amine or polyamine, or combinations thereof.
1.17. Any of the preceding compositions, wherein the amine base is a polyamine (e.g., a monoamine base, a diamine base and/or a triamine base).
1.18. Any of the preceding compositions, wherein the amine base is a monoamine base.
1.19. Any of the preceding compositions, wherein the amine base is a diamine base.
1.20. Any of the preceding compositions, wherein the amine base is a triamine base.
1.21. Any of the preceding compositions, wherein the amine base comprises one or more of N′-octadecyltrimethylendiamine-N,N,N′-tris(2-ethanol), and/or oleyldiamine ethoxylate and/or N-octadeca-9-enylamine, and/or alkyl trihydroxyethyl propylenediamine.
1.22. Any of the preceding compositions, wherein the amine base is N′-octadecyltrimethylendiamine-N,N,N′-tris(2-ethanol).
1.23. Any of the preceding compositions, wherein the amine base is N-octadeca-9-enylamine.
1.24. Any of the preceding compositions, wherein the amine base and fluoride form amine fluoride in situ.
1.25. Any of the preceding compositions, wherein the amine fluoride is olaflur (N′-octadecyltrimethylenediamine-N,N,N′-tris(2-ethanol)-dihydrofluoride).
1.26. Any of the preceding compositions, wherein the amine fluoride is present in an amount of from 0.5% to 1.5%, e.g., from 0.5% to 1.2%, from 0.5% to 1%, from 0.7% to 1.5%, from 0.7% to 1.2%, from 0.7% to 1%, from 0.8% to 1.5%, from 0.8% to 1.2%, from 0.8% to 1%, from 0.85% to 0.95%, or about 0.9% by weight of the composition.
1.27. Any of the preceding compositions, wherein the composition comprises olaflur and coco glucoside.
1.28. Any of the preceding compositions, wherein the composition comprises olaflur in an amount of from 0.5% to 1.5 by weight of the composition and coco glucoside in an amount of from 0.1% to 1% by weight of the composition.
1.29. Any of the preceding compositions, wherein the composition comprises olaflur in an amount of from 0.8% to 1.2% by weight of the composition and coco glucoside in an amount of from 0.2% to 0.4% by weight of the composition.
1.30. Any of the preceding compositions, wherein the composition comprises olaflur in an amount of from 0.8% to 1%, e.g., about 0.9% by weight of the composition and coco glucoside in an amount of from 0.25% to 0.3%, e.g., about 0.27% by weight of the composition.
1.31. Any of the preceding compositions, wherein the composition is free or substantially free of sodium lauryl sulfate.
1.32. Any of the preceding compositions, wherein the composition is free or substantially free of alkyl sulfate salts.
1.33. Any of the preceding compositions, wherein the composition is free or substantially free of anionic surfactants.
1.34. Any of the preceding compositions, wherein the composition does not comprise any surfactant other than the alkyl glucoside and amine fluoride.
1.35. Any of the preceding compositions comprising a humectant, e.g., selected from glycerin, sorbitol, propylene glycol, polyethylene glycol, xylitol, and mixtures thereof.
1.36. Any of the preceding compositions, wherein the composition comprises one or more soluble phosphate salts, e.g., selected from tetrasodium pyrophosphate (TSPP), sodium tripolyphosphate (STPP) and a combination thereof.
1.37. Any of the preceding compositions, wherein the composition comprises a basic amino acid in free or salt form, optionally wherein the basic amino acid comprises arginine.
1.38. Any of the preceding compositions, wherein the composition comprises a zinc ion source.
1.39. Composition 1.38, wherein the zinc ion source is selected from the group consisting of zinc oxide, zinc sulfate, zinc chloride, zinc citrate, zinc lactate, zinc gluconate, zinc malate, zinc tartrate, zinc carbonate, zinc phosphate and a combination thereof.
1.40. Any of Composition 1.38 to 1.39, wherein the zinc ion source is selected from the group consisting of zinc oxide, zinc citrate, and a combination thereof, optionally wherein the zinc ion source is a combination of zinc oxide and zinc citrate.
1.41. Any of the preceding compositions comprising a sweetener, optionally wherein the sweetener is saccharin.
1.42. Any of the preceding compositions comprising a breath freshener.
1.43. Any of the preceding compositions comprising a fragrance.
1.44. Any of the preceding compositions comprising a flavorant.
1.45. Any of the preceding compositions comprising a pigment.
1.46. Any of the preceding compositions comprising a preservative.
1.47. Any of the preceding compositions, wherein the composition comprises water.
1.48. Any of the preceding compositions which is substantially free of ethanol, e.g., having less than 0.5% ethanol by weight.
1.49. Any of the preceding compositions, wherein the mouthwash is a micellar mouthwash.

The mouthwash composition of the present invention comprises an amine fluoride or amine base plus fluoride. As used herein, the term “amine base” may refer to a primary amine base, a secondary amine base or a tertiary amine base. “Primary amine base” refers to a compound containing at least one amine in which the nitrogen atom is directly bonded to one carbon of any hybridization, except for carbonyl group carbons. “Secondary amine base” refers to a compound containing at least one amine in which the nitrogen atom is directly bonded to two carbons of any hybridization, except for carbonyl group carbons. “Tertiary amine base” refers to a compound containing at least one amine in which the nitrogen atom is directly bonded to three carbons of any hybridization, except for carbonyl group carbons. “Amine base” may be used to refer to compounds containing a plurality of primary, secondary and/or tertiary amine groups (e.g., a tertiary polyamine). In particular, the term “amine base” excludes acid addition salts (e.g., hydrochloride salts and hydrofluoride salts), and thus refers to the free base form of the molecule. In some embodiments, the amine base is selected from N′-octadecyltrimethylendiamine-N,N,N′-tris(2-ethanol), oleyldiamine ethoxylate, N-octadeca-9-enylamine, alkyl trihydroxyethyl propylenediamine, and a combination thereof. Hydrofluoride derivatives of amines are referred to herein as “amine fluorides.” In methods for the production or manufacture of a composition containing an amine fluoride, an amine base may be a precursor to forming the amine fluoride. In certain embodiments, the amine fluoride is olaflur (N′-octadecyltrimethylenediamine-N,N,N′-tris(2-ethanol)-dihydrofluoride).

In some embodiments, the amine base and fluoride form amine fluoride in situ. As used herein, the term “in situ” is used to refer to the formation of a chemical product (e.g., amine fluoride) in the mouthwash composition. For example, the reaction may be a salination reaction carried out by mixing an amine with a fluoride source and an acid, thus creating an amine fluoride and a salt.

In some embodiments, the amine fluoride is present in an amount of from 0.5% to 1.5%, e.g., from 0.5% to 1.2%, from 0.5% to 1%, from 0.7% to 1.5%, from 0.7% to 1.2%, from 0.7% to 1%, from 0.8% to 1.5%, from 0.8% to 1.2%, from 0.8% to 1%, from 0.85% to 0.95%, or about 0.9% by weight of the composition.

The mouthwash composition of the present invention comprises an alkyl glucoside. Alkyl glucoside is a compound produced by combining a sugar such as glucose with a fatty alcohol. Alkyl refers to unbranched or branched carbon chain. In some embodiments, the alkyl group is unbranched. The alkyl group may be saturated or unsaturated. In some embodiments, the alkyl group is saturated. For example, the structure of decyl glucoside is shown below:

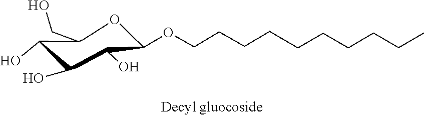

Alkyl glucoside may be C₈₋₂₅ alkyl glucoside, e.g., C₈₋₁₈ alkyl glucoside, C₁₀₋₁₈ alkyl glucoside or C₁₀₋₁₆ alkyl glucoside. In some embodiments, the alkyl glucoside is selected from coco glucoside, decyl glucoside, caprylyl/capryl glucoside, lauryl glucoside, octyl glucoside, cetearyl glucoside, cetyl glucoside, hexadecyl glucoside, arachidyl glucoside, and a combination thereof. In certain embodiments, the alkyl glucoside is coco glucoside.

In some embodiments, the alkyl glucoside is present in an amount of from 0.1% to 2.5%, e.g., from 0.1% to 1%, from 0.1% to 0.6%, from 0.1% to 0.5%, from 0.1% to 0.4%, from 0.1% to 0.3%, from 0.2% to 0.4%, from 0.2% to 0.3%, from 0.25% to 0.3%, or about 0.27% by weight of the composition.

In some embodiments, the mouthwash composition of the present invention is free or substantially free of sodium lauryl sulfate. In some embodiments, the composition is free or substantially free of alkyl sulfate salts. In some embodiments, the composition is free or substantially free of anionic surfactants. In some embodiments, the composition does not comprise any surfactant other than the alkyl glucoside and amine fluoride.

The mouthwash composition of the present invention comprises water. Water employed in the preparation of the mouthwash composition should be deionized and free of organic impurities. Water may make up the balance of the mouthwash composition. In some embodiments, water is present in an amount of from 70% to 99%, e.g., 80% to 99%, 90% to 99%, by weight of the composition. This amount of water includes the free water which is added plus that amount which is introduced with other components of the oral care composition, such as with sorbitol.

The mouthwash composition of the present invention may include one or more humectants. Humectants can reduce evaporation and also contribute towards preservation by lowering water activity and can also impart desirable sweetness or flavor to compositions. Illustrative humectants may be or include, but are not limited to, glycerin, propylene glycol, polyethylene glycol, sorbitol, xylitol, or the like, or any mixture or combination thereof. In a preferred embodiment, the orally acceptable vehicle may be or include, but is not limited to, glycerin or sorbitol. In some embodiments, the humectant is selected from glycerin, sorbitol and a combination thereof.

The mouthwash composition of the present invention may include a basic amino acid in free or salt form. The basic amino acids which can be used in the compositions include not only naturally occurring basic amino acids, such as arginine, lysine, and histidine, but also any basic amino acids having a carboxyl group and an amino group in the molecule, which are water-soluble and provide an aqueous solution with a pH of about 7 or greater. Accordingly, basic amino acids include, but are not limited to, arginine, lysine, citrullene, ornithine, creatine, histidine, diaminobutanoic acid, diaminopropionic acid, salts thereof or combinations thereof. In a particular embodiment, the basic amino acids are selected from arginine, lysine, citrullene, and ornithine. The basic amino acids of the oral care composition may generally be present in the L-form or L-configuration. The basic amino acids may be provided as a salt of a di- or tri-peptide including the amino acid. In some embodiments, at least a portion of the basic amino acid present in the mouthwash composition is in the salt form. In some embodiments, the basic amino acid is arginine, for example, L-arginine, or a salt thereof. Arginine may be provided as free arginine or a salt thereof. For example, Arginine may be provided as arginine phosphate, arginine hydrochloride, arginine sulfate, arginine bicarbonate, or the like, and mixtures or combinations thereof. The basic amino acid may be provided as a solution or a solid. For example, the basic amino acid may be provided as an aqueous solution. In some embodiment, the amino acid includes or is provided by an arginine bicarbonate solution. For example, the amino acid may be provided by an about 40% solution of the basic amino acid, such as arginine bicarbonate or alternatively called as arginine carbamate.

The mouthwash composition of the present invention may include a zinc ion source. The zinc ion source may be or include a zinc ion and/or one or more zinc salts. For example, the zinc salts may at least partially dissociate in an aqueous solution to produce zinc ions. Illustrative zinc salts may include, but are not limited to, zinc lactate, zinc oxide, zinc chloride, zinc phosphate, zinc citrate, zinc acetate, zinc borate, zinc butyrate, zinc carbonate, zinc formate, zinc gluconate, zinc glycerate, zinc glycolate, zinc picolinate, zinc proprionate, zinc salicylate, zinc silicate, zinc stearate, zinc tartrate, zinc undecylenate, and mixtures thereof. In some embodiments, the zinc ion source is selected from zinc oxide, zinc citrate, and a combination thereof.

The mouthwash composition of the present invention may include a preservative. Suitable preservatives include, but are not limited to, sodium benzoate, potassium sorbate, methylisothiazolinone, paraben preservatives, for example methyl p-hydroxybenzoate, propyl p-hydroxybenzoate, and mixtures thereof. In certain embodiments, the preservative is sodium benzoate.

The mouthwash composition of the present invention may include a sweetener such as, for example, saccharin, for example sodium saccharin, acesulfam, neotame, cyclamate or sucralose; natural high-intensity sweeteners such as thaumatin, stevioside or glycyrrhizin; or such as sorbitol, xylitol, maltitol or mannitol. In certain embodiments, the sweetener is saccharin, e.g., sodium saccharin.

The mouthwash composition of the present invention may include a flavoring agent. Suitable flavoring agents include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole.

The mouthwash composition of the present invention may include one or more pH modifying agents. For example, the oral care composition may include one or more acidifying agents and/or one or more basifying agents configured to reduce and/or increase the pH thereof, respectively. Illustrative acidifying agents and/or one or more basifying agents may be or include, but are not limited to, an alkali metal hydroxide, such as sodium hydroxide and/or potassium hydroxide, citric acid, hydrochloric acid, or the like, or combinations thereof.

The mouthwash composition of the present invention may also include one or more buffering agents configured to control or modulate the pH within a predetermined or desired range. Illustrative buffering agents may include, but are not limited to, sodium bicarbonate, sodium phosphate, sodium carbonate, sodium acid pyrophosphate, sodium citrate, and mixtures thereof. Sodium phosphate may include monosodium phosphate (NaH₂PO₄), disodium phosphate (Na₂HPO₄), trisodium phosphate (Na₃PO₄), and mixtures thereof. In a typical embodiment, the buffering agent may be anhydrous sodium phosphate dibasic or disodium phosphate and/or sodium phosphate monobasic. In another embodiment, the buffering agent includes anhydrous sodium phosphate dibasic or disodium phosphate, and phosphoric acid (e.g., syrupy phosphoric acid; 85%-Food Grade).

The mouthwash composition of the present invention may include anticalculus agents. Illustrative anticalculus agents may include, but are not limited to, phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates. In some embodiments, the anticalculus agent includes tetrasodium pyrophosphate (TSPP), sodium tripolyphosphate (STPP), or a combination thereof.

The mouthwash composition of the present invention may include an antioxidant. Any orally acceptable antioxidant may be used, including, but not limited to, butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, or the like, or combinations and mixtures thereof.

All ingredients for use in the compositions described herein should be orally acceptable. As used herein, “orally acceptable” may refer any ingredient that is present in a composition as described in an amount and form which does not render the composition unsafe for use in the oral cavity.

In another aspect, the present invention provides a method to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity, comprising applying an effective amount of any of mouthwash compositions as disclosed herein to the oral cavity of a subject in need thereof.

In another aspect, the invention provides the use of any of mouthwash compositions as disclosed herein to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity, in a subject in need thereof.

In another aspect, the invention provides the use of an alkyl glucosides in a mouthwash composition comprising an amine fluoride, e.g., any of mouthwash compositions as disclosed herein for improving micellar properties of the mouthwash composition, e.g., decreasing critical micelle concentration (CMC) of the amine fluoride in the composition and/or decreasing micelle (colloid particle) size.

### EXAMPLES

Two solutions having the formulas as indicated in Table 1 are prepared.

TABLE 1

| | Formulation A | Formulation B |
|---|---|---|
| | (pH = 4.9) | (pH = 4.75) |
| Amine fluoride (Olaflur) | 0.9% | 0.9% |
| Coco glucoside (50%) | 0.54% | 0% |
| PEG-40 hydrogenated castor oil (90%) | 0% | 0.3% |
| water | q.s. | q.s. |

Formulation contains an amine fluoride (Olaflur) an coco glucoside, while Formulation B contains Olaflur and PEG-40 hydrogenated castor oil, which is used as a surfactant in a commercial mouthwash (Elmex) formulation. The Elmex mouthwash contains 0.89% Olaflur and 0.27% PEG-40 hydrogenated castor oil. Surfactants form micelles in a solution once the surfactant concentration reaches a threshold value, called critical micelle concentration (CMC). The CMC of Olaflur in Formulations A and B as well as Elmex mouthwash is determined as the inflection point in a surface tension isotherm, where the slope deviates from the linearly decreasing section. Surface tension is measured with an Attension tensiometer at room temperature. The results are shown in Table 2.

TABLE 2

| Formulation | CMC of Olaflur (wt %) |
|---|---|
| Elmax | 1 |
| A | 0.005 |
| B | 0.09 |

The CMC of Formulation A is about 0.005%, while the CMC of Formulation B is 0.09%. In other words, the concentration of Olaflur in Formulation A is 180 times higher than its CMC, while the concentration of Olaflur in Formulation B is 10 times higher than its CMC. The lower CMC of Formulation A ensures a greater population of micelles. These results show that the combination of amine fluoride and coco glucoside is a much improved alternative to amine fluoride/PEG-40 hydrogenated castor oil to form micelles in a mouthwash formulation. The CMC of the Elmex mouthwash is determined to be about 1%, although the inflection point of the surface tension curve is not obvious in the Elmex mouthwash.

Next, the colloid particle (micelle) size in Formulations A and B as well as Elmex mouthwash is examined. The results are shown in Table 3.

TABLE 3

Micelle size (nm)

| Sample Name | mean | standard deviation |
|---|---|---|
| Elmex | 12.2 | n/a |
| Olafur | 6.6 | 0.3 |
| Coco glucosides | 23.7 | 0.1 |
| PEG-40 castor oil | 16 | 0.1 |
| Formulation A | 3.3 | 0.1 |
| Formulation B | 14.1 | 0.4 |

The micelle size of Formulation A is around 3.3 nm, remarkably lower than the micelle size of individual amine fluoride (6.6 nm) and individual coco glucoside (23.7 nm), suggesting the strong synergistic interaction between these two components. In contrast, the micelle size of Formulation B is around 14.1 nm, close to that of PEG-40 hydrogenated castor oil (16.0 nm). Although the inflection point of the surface tension curve is not obvious in the Elmex mouthwash, dynamic light scattering detects colloid particles with 12.2 nm diameter, which are likely solubilized oil droplets in the mouthwash product. With the smallest particle size (amine fluoride/coco glucoside<amine fluoride<amine fluoride/PEG-40 hydrogenated castor oil), amine fluoride/cocoyl glucoside complex is most mobile and can likely reach smaller spaces that others cannot reach. This feature would likely enhance the delivery of active encapsulated in the micelle core, and also enhance availability of amine fluoride, therefore improving its antibacterial efficacy.

2D NOESY experiments are carried out to further elucidate the interaction between amine fluoride and other surfactant (alkyl glucoside or PEG-40 hydrogenated castor oil). The results show cross peaks at around 2-3 ppm indicating the presence of interaction between hydrocarbon chains of coco glucoside and the headgroup of amine fluoride. However, no obvious interaction is found in the formulation B. The diffusion rate of each ingredient is further measured by NMR. The results are shown in Table 4.

TABLE 4
Diffusion rate D determined by NMR

| | D in | D in | D in |
|---|---|---|---|
| | individual | Formulation | Formulation |
| Ingredients | solution, cm²/s | A, cm²/s | B, cm²/s |
| Amine Fluoride (Olaflur) | 4.84 × 10⁻⁶ | 2.27 × 10⁻⁶ | 4.33 × 10⁻⁶ |
| Coco glucoside | 2.82 × 10⁻⁶ | 2.08 × 10⁻⁶ | — |
| PEG-40 hydrogenated | 2.08 × 10⁻⁶ | — | 2.05 × 10⁻⁶ |
| castor oil | | | |

The results show that when amine fluoride an coco glucoside are mixed in Formulation A, the molecular diffusion of two ingredients reduces to almost the same level, suggesting they form aggregates. In contrast, the diffusion of amine fluoride and PEG-40 hydrogenated castor oil in Formulation B does not change significantly, compared to that in the individual solution. These findings are consistent with the NOESY results, suggesting that amine fluoride and alkyl glucoside interact with each other to form aggregates.

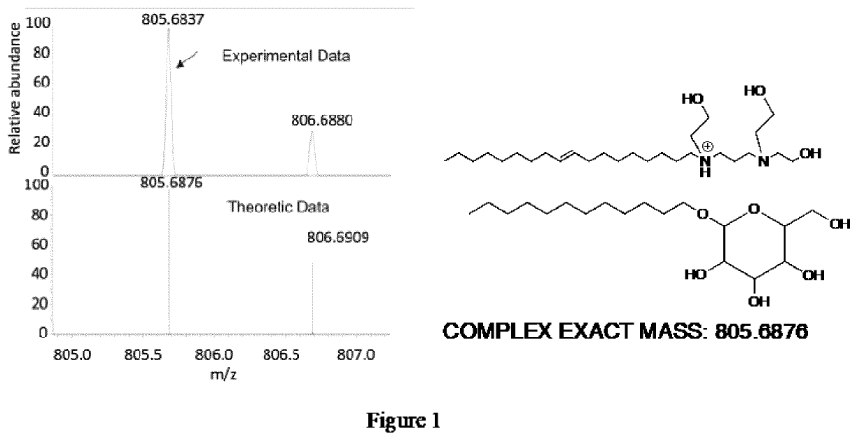

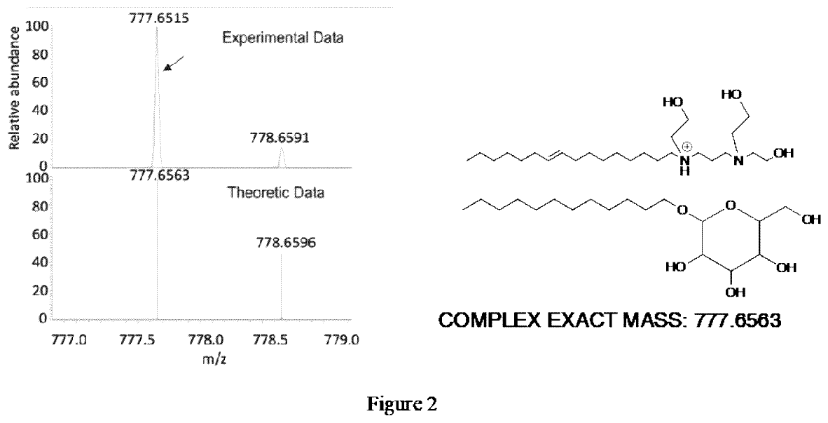

Liquid chromatography-mass spectrometry (LCMS) analysis is carried out with a Q-Exactive™ Orbitrap™ mass spectrometer (Thermo Scientific, SanJose, USA) equipped with a heated electrospray ionization source (HESI-II) for the mixture of amine fluoride and coco glucoside. Samples are analyzed in full scan MS mode under negative polarity condition using electron spray ionization. The results of the LCMS analysis are shown in FIGS. 1 and 2. As shown in FIGS. 1 and 2, the complexes formed between amine and coco glucoside are detected, with the high resolution mass spectra consistent with theoretic data expected from dodecyl glucoside plus amine base (C18 amine and C16 amine), respectively. These results confirm that amine fluoride interact with coco glucoside to form a complex.

While the present disclosure has been described with reference to embodiments, it will be understood by those skilled in the art that various modifications and variations may be made therein without departing from the scope of the present disclosure as defined by the appended claims.

1. A mouthwash composition comprising an amine fluoride or amine base plus fluoride and an alkyl glucoside.

2. The composition according to claim 1, wherein the alkyl glucoside is C₈₋₂₅ alkyl glucoside.

3. The composition according to any of preceding claims, wherein the alkyl glucoside is selected from coco glucoside, decyl glucoside, caprylyl/capryl glucoside, lauryl glucoside, octyl glucoside, cetearyl glucoside, cetyl glucoside, hexadecyl glucoside, arachidyl glucoside, and a combination thereof.

4. The composition according to any of preceding claims, wherein the alkyl glucoside is coco glucoside.

5. The composition according to any of preceding claims, wherein the alkyl glucoside is present in an amount of from 0.1% to 2.5% by weight of the composition.

6. The composition according to any of preceding claims, wherein the alkyl glucoside is present in an amount of from 0.2% to 0.4% by weight of the composition.

7. The composition according to any of preceding claims, wherein the amine base is a linear or branched fatty amine or polyamine.

8. The composition according to any of the preceding claims, wherein the amine base is a saturated or unsaturated C₁₂₋₂₀ alkyl amine base or a saturated or unsaturated C₁₂₋₂₀ alkyl polyamine base.

9. The composition according to any of the preceding claims, wherein the amine base is a myristyl, palmityl, linoleyl, oleyl, or stearyl amine or polyamine, or N′-octadecyltrimethylendiamine-N,N,N′-tris(2-ethanol), or N-octadeca-9-enylamine, and combinations thereof.

10. The composition according to any of the preceding claims, wherein the amine base and fluoride form amine fluoride in situ.

11. The composition according to any of the preceding claims, wherein the amine fluoride is olaflur (N′-octadecyltrimethylenediamine-N,N,N′-tris(2-ethanol)-dihydrofluoride).

12. The composition according to any of the preceding claims, wherein the amine fluoride is present in an amount of from 0.5% to 1.5% by weight of the composition.

13. The composition according to any of the preceding claims, wherein the amine fluoride is present in an amount of from 0.8% to 1.2% by weight of the composition.

14. The composition according to any of the preceding claims, wherein the composition comprises olaflur in an amount of from 0.8% to 1.2% by weight of the composition and coco glucoside in an amount of from 0.2% to 0.4% by weight of the composition.

15. The composition according to any of the preceding claims, wherein the composition is free or substantially free of sodium lauryl sulfate.

16. The composition according to any of the preceding claims, wherein the composition is free or substantially free of anionic surfactants.

17. Use of an alkyl glucosides in a mouthwash composition comprising an amine fluoride for improving micellar properties of the mouthwash composition.

18. The use according to claim 17, wherein the improvement of micellar properties of the mouthwash composition comprises decreasing critical micelle concentration (CMC) of the amine fluoride in the composition and/or decreasing micelle (colloid particle) size.
